(19) United States
(12) Patent Application Publication
(10) Pub. No.: US 2021/0251823 A1
(43) Pub. Date: Aug. 19, 2021

(54) ABSORBENT ARTICLE WITH A THREE-DIMENSIONAL SUBSTRATE

(71) Applicant: The Procter & Gamble Company, Cincinnati, OH (US)

(72) Inventors: Yi YUAN, Beijing (CN); Meng CHEN, Beijing (CN); Hui LIU, Beijing (CN); Koichi MORIMOTO, Beijing (CN); Gueltekin ERDEM, Beijing (CN); Li LIU, Beijing (CN)

(21) Appl. No.: 17/178,299

(22) Filed: Feb. 18, 2021

Related U.S. Application Data
(63) Continuation of application No. PCT/CN2020/075655, filed on Feb. 18, 2020.

(51) Int. Cl. A61F 13/514; A61F 13/15; A61F 13/512; A61F 13/539

(52) U.S. Cl. CPC A61F 13/51476; A61F 13/15203; A61F 13/51496; A61F 13/5123; A61F 2013/530481; A61F 2013/53908; A61F 2013/15406; A61F 2013/15414; A61F 13/539

(57) ABSTRACT

The present disclosure is directed to an absorbent article comprising a liquid pervious topsheet, a backsheet, an absorbent core disposed between the topsheet and the backsheet, and an outer cover forming at least part of a garment-facing surface of the absorbent article, the outer cover comprising a three-dimensional substrate, wherein the three-dimensional substrate comprises a first layer and a second layer, and a plurality of protrusions where the first layer is unbonded to the second layer, and wherein the absorbent article has a Qmax-wet of 1300 W/m² or less as measured according to Qmax Test.

### CROSS REFERENCE TO RELATED APPLICATIONS

This application is a continuation, under 35 U.S.C. § 120, of Application No. PCT/CN2020/075655, filed on Feb. 18, 2020, which is herein incorporated by reference in its entirety.

### FIELD

The present disclosure is for an absorbent article such as a baby diaper, a training pant, a feminine hygiene sanitary napkin or an adult incontinence product. The absorbent article of the present disclosure comprises a three-dimensional substrate disposed on the garment-facing side of the article.

### BACKGROUND

Absorbent articles for personal hygiene, such as disposable diapers for infants, training pants for toddlers, adult incontinence undergarments and/or sanitary napkins are designed to absorb and contain body exudates, in particular large quantities of urine, runny bowel movement (BM) and/or menses.

These absorbent articles comprise several layers providing different functions. A liquid permeable topsheet is disposed closest to the wearer's skin and should be capable of quickly absorbing the excreted fluid. A backsheet is disposed on the opposed, garment-facing side of the article. Other components of absorbent articles are well known, and include in particular an absorbent core disposed between the topsheet and the backsheet to absorb and retain the excreted fluids.

A backsheet is a portion touched and observed by the wearer or the caregiver upon use, and thus its properties are most associated with the function and quality of the article. The backsheet typically comprises a liquid impermeable material. Many absorbent articles in the market further comprises a nonwoven outermost layer. A nonwoven outermost layer is generally expected to be soft, anti-fuzz and feel dry even when absorbent articles are wet. When absorbent articles absorb liquid, the externally-facing side of the absorbent article may feel colder than the room temperature and be perceived as wet. A thinner, low basis weight nonwoven outermost layer typically has a lower thermal resilience value in comparison with a thicker, high basis weight nonwoven layer, therefore an absorbent article with it tends to deliver a cold and damp feeling when the absorbent article is wet. Employment of a thick nonwoven outer most layer may mitigate the cold and damp feeling, but it may not be preferred as it makes the absorbent article thick and stiff and causes a cost increase.

There is a need to provide an absorbent article having a relatively high thermal resilience to mitigate a cold and damp feel when it is wet without increase an absorbent article thickness.

There is a need to provide an absorbent article which has little cold and damp feel even when it is wet without a material cost increase.

### SUMMARY

The present disclosure is directed to an absorbent article an outer cover, an outermost layer, comprising a three-dimensional substrate, wherein the outer cover comprises a first layer, a second layer, and a plurality of protrusions, and wherein the absorbent article has a Qmax-wet of about 1300 W/m² or less as measured according to Qmax Test described herein.

The article is illustrated in the Figures as a taped diaper. For ease of discussion, the absorbent article and the three-dimensional substrate will be discussed with reference to the numerals referred to in these Figures. The Figures and detailed description should however not be considered limiting the scope of the claims, unless explicitly indicated otherwise, and the invention disclosed herein is also used in a wide variety of absorbent article forms.

### BRIEF DESCRIPTION OF THE DRAWINGS

FIG. 1 is a schematic cross-sectional view of an exemplary substrate for an outer cover.

FIG. 2 is a schematic cross-sectional view of an alternative substrate for an outer cover.

FIG. 3 is a schematic illustration of a process for making a substrate as in FIG. 2.

FIG. 4 is a schematic plan view of an exemplary absorbent article according to the present disclosure.

FIG. 5 is a lateral cross-section view of the absorbent article of FIG. 4.

FIGS. 6A and 6B are schematic views of sample preparation and an exemplary apparatus used for the Qmax Test.

FIGS. 7A-7C are schematic views of sample preparation and an exemplary apparatus used for the Dampness Difference Test.

FIG. 8 is a photograph of a three-dimensional substrate (10 cm by 10 cm).

FIG. 9 is a photograph of another three-dimensional substrate (10 cm by 10 cm).

FIG. 10 is a photograph of another three-dimensional substrate (10 cm by 10 cm).

FIG. 11 is a photograph of another three-dimensional substrate (10 cm by 10 cm).

FIG. 12 is a photograph of another three-dimensional substrate (10 cm by 10 cm).

### DETAILED DESCRIPTION

Various non-limiting forms of the present disclosure will now be described to provide an overall understanding of the principles of the structure, function, manufacture, and use of an absorbent article according to the present disclosure. One or more examples of these non-limiting embodiments are illustrated in the accompanying drawings. The features illustrated or described in connection with one non-limiting form may be combined with the features of other non-limiting forms. Such modifications and variations are intended to be included within the scope of the present disclosure.

### Definitions of Terms

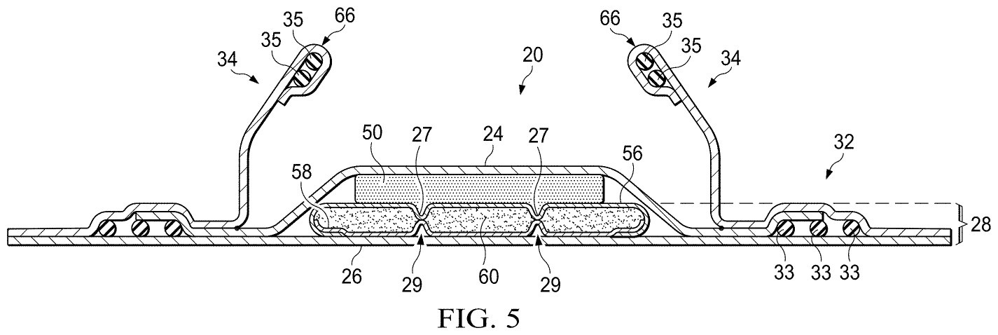

The term “absorbent article” as used herein refers to disposable products such as taped diapers, diapers having a closed waist opening (pants), feminine hygiene sanitary napkins and the like, which are placed against or in proximity to the body of the wearer to absorb and contain bodily exudates such as urine, feces and menses discharged from the body. Typical absorbent articles comprise a topsheet, a backsheet, an absorbent core, an acquisition layer and other components. A liquid permeable topsheet forms at least a portion of the wearer-facing side of the article, and a backsheet forms at least a portion, and typically the whole, of the garment-side of the article. The articles may be provided with fastening elements, such as tapes (taped diapers) or may be provided already pre-formed with a waist opening and a pair of leg openings as in an underwear (pant diapers). The absorbent articles may be for use with babies, infants, women or incontinent adults. Typical features of absorbent articles are discussed further below, and in relation with the illustrated taped diaper in FIGS. 4 and 5, which is of course for illustration purpose only and not limiting the scope of the inventions, unless specifically indicated otherwise.

Any preferred or exemplary embodiments described below are not limiting the scope of the claims, unless specifically indicated to do so. The words “typically”, “normally”, “advantageously” and the likes also qualify features which are not intended to limit the scope of the claims unless specifically indicated to do so.

“Natural fibers” refers to elongated substances produced by plants and animals and includes animal-based fibers and plant-based fibers, as those categories are described herein. Natural fibers, as that term is used herein, include fibers harvested without any post-harvest treatment step as well as those having a post-treatment step, such as, for example, washing, scouring, bleaching.

The term “nonwoven” as used herein refers to a manufactured material, web, sheet or batt of directionally or randomly oriented fibers, bonded by friction, and/or cohesion and/or adhesion, excluding paper and products which are woven, knitted, tufted, stitch-bonded, incorporating binding yarns or filaments, or felted by wet milling, whether or not additionally needled. The fibers may be of natural or man-made origin. The fibers may be staple or continuous filaments or be formed in situ. The porous, fibrous structure of a nonwoven may be configured to be liquid permeable or impermeable, as desired.

### Three-Dimensional Substrate

The three-dimensional substrate (herein also more simply referred to as “the substrate layer” or “the substrate”) disclosed herein comprises a first layer and a second layer. While the first layer and the second layer will be discussed individually in greater details in dedicated sections further below, this section will briefly discuss the substrate comprising these two layers, as a whole.

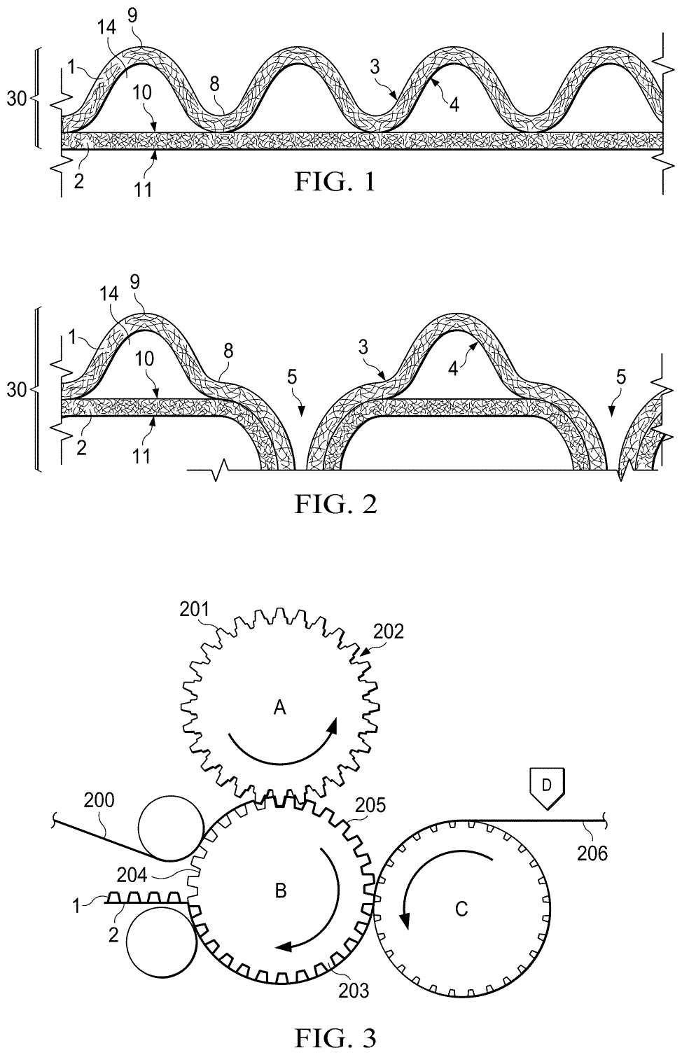

FIGS. 1 and 2 schematically show cross-sections of two exemplary substrates 30 according to the invention. The substrate comprises a first layer 1 and a second layer 2. The first layer 1 and the second layer 2 are disposed in a face to face relationship in the substrate 30. The first layer 1 comprises a first surface 3 and a second surface 4. The second layer 2 comprises a first surface 10 and a second surface 11. The first surface 10 of the second layer 2 faces the second surface 4 of the first layer 1. The first surface 3 of the first layer 1 and the first surface 10 of the second layer 2 are typically facing outwardly (externally), towards the wearer-facing side of the article, while the second surface 4 of the first layer 1 and the second surface 11 of the second layer 2 are typically facing inwardly (internally), when the substrate is incorporated in an absorbent article. The first layer 1 comprises a plurality of protrusions 9 which are unbonded to the second layer 2, so that the first layer 1 is intermittently bonded to the second layer 2. The protrusions 9 provide a three-dimensional profile to the first layer 1 and more generally to the substrate 30 as a whole.

The first layer 1 and the second layer 2 may be generally contiguous in the horizontal plane, but it is not excluded that the second layer may be wider or longer than the first layer. In this example, the first layer may have a smaller area disposed in the central region of the article relative to the second layer. The protrusions may also present only in a selected area of the substrate that is smaller than the overall surface of the substrate.

The first layer 1 and the second layer 2 forming the substrate 30 are intermittently bonded by technologies known in the art. For example, the first layer 1 and the second layer 2 can be laminated by either adhesive or thermally bonded means, where thermal bonding includes but is not restricted to technologies such as ultrasonic bonding, cold pressure bonding, and hot pressure bonding. When the first layer comprises a relatively high amount of natural fibers, bonding the first and second layer to each other using heat may be difficult as the natural fibers do not become tacky or melt upon exposure to heat. The first layer 1 is thus attached to the second layer 2 in bonding areas by hot-melt adhesive.

The substrate 30 may comprise a plurality of apertures 5. Referring to FIG. 1 a substrate 30 according to the present invention may not comprise apertures. Referring to FIG. 2, a substrate 30 according to the present invention may further comprise apertures 5. The apertures 5 in the substrate can enhance the overall air flow and breathability of the absorbent article.

In some embodiments referring to FIGS. 1 and 2, in a substrate perspective, the protrusions can have hollow space and thus comprise an inside void volume 14 which is the portion of the protrusion which does not comprise any fibers, or at least very little fibers. The presence of protrusions can typically improve the softness of the substrate, and thus of the garment-facing side of the article on which it is disposed.

The void volume 14 may have a width WD, which is the maximum interior width measured between two side walls of the inner protrusion (or which is the maximum diameter of the side wall of the inner protrusion when the distal portion has a substantially circular shape). The width WD of the protrusions 9 may for example range from 0.5 mm to 15 mm or from 0.5 mm to 10 mm or from 0.5 mm to 5 mm or from 0.5 mm to 3 mm. Measurements of the dimensions of the width of the height of the protrusions 9 can be made on a photomicrograph.

The substrate including the first layer and second layer may typically have a basis weight from 15 g/m² to 80 g/m², or from 15 g/m² to 60 g/m², or from 20 g/m² to 50 g/m². The first layer may have a basis weight of from 10 g/m² to 50 g/m², or from 15 g/m² to 40 g/m². The second layer may have a basis weight of from 5 g/m² to 50 g/m², or from 7 g/m² to 30 g/m², or from 7 g/m² to 20 g/m². The second layer may have a basis weight that is at least 5 g/m² lower, or at least 10 g/m² lower, than the basis weight of the first layer. It has been found that such relatively low basis weight layers are sufficient to facilitate stabilization of the three-dimensional configuration of the first layer. The substrate typically comprises these two layers only, but it is not excluded that a third or more layers may also be present in the substrate.

The substrate may be in a close contact with adjacent layers of the absorbent article.

Protrusions

The plurality of protrusions 9 imparts a three-dimensional shape to the first layer and the substrate.

Viewed from a cross-sectional view as in FIGS. 1 and 2, i.e. in a Z-direction, the protrusions may have any suitable shapes, including but not limited to: cylindrical, bulbous-shaped, conical-shaped, mushroom shaped and heart-shaped. Viewed from above, the majority of the protrusions may have any suitable shapes, including but not limited to: circular, diamond-shaped, round diamond-shaped, U.S. football-shaped, oval-shaped, clover-shaped, triangular-shaped, tear-drop shaped and elliptical-shaped protrusions. The protrusions may have a Z-directional height in the range from about 0.1 mm to about 6.0 mm, or from about 0.3 mm to about 4.0 mm, or from about 0.5 mm to about 3.0 mm, measured from the land areas 8 to the top of the protrusion.

The protrusions 9 may be uniformly distributed on the first surface 3 of the first layer 1. The protrusions 9 may be provided throughout the complete surface of the first layer 1 or may only be provided in a portion of the first layer 1. Two or more adjacent protrusions 9 are separated by one or more land areas 8 and optionally one or more apertures 5. The protrusions 9 may be surrounded by a plurality of land areas 8 and/or a plurality of apertures 5. The land areas 8 may be substantially flat areas. In some embodiments, the land areas 8 are flat areas. The land areas 8 may fully surround the protrusions 9. The optional apertures 5 may be located between the protrusions and will be discussed further below. The protrusions may extend upwardly from land areas 8 that form a base and have an opposed distal portion from the land areas 8 forming a peak. The base of the protrusions 9, where each protrusion starts to protrude outwardly from the land areas 8, define a perimeter, which for circular protrusions is the circumference.

In some embodiments referring to FIGS. 1 and 2, the protrusions are formed on the first layer 1, and the second layer 2 may be free of protrusions. The area of the second layer which coincides with the protrusions of the first layer, may be substantially flat, or may be flat.

The first layer 1 and the second layer 2 may be joined with each other at the land areas 8, at least partially, between the protrusions 9 and/or at the apertures 5. The first layer 1 and the second layer 2 are typically not in contact in the areas of the protrusions 9.

First Layer

The substrate disclosed herein, referring to FIGS. 1 and 2, comprises a first layer 1, which is typically disposed more outwardly than the second layer 2, relative to an absorbent article, and to form at least part of an outermost layer of the absorbent article.

The first layer may be any type of web conventionally used in the art. For example, it may be a nonwoven web of natural fibers, synthetic fibers or a combination of natural and synthetic fibers. Several examples of nonwoven materials suitable for use as a first layer include, but are not limited to: spunbonded nonwovens; carded nonwovens; air through bonded carded nonwovens; spunlace nonwovens; needle punched nonwovens and nonwovens with relatively specific properties to be able to be readily deformed.

The first layer may comprise synthetic fibers, or mixed with the natural fibers. Synthetic fibers may be selected from the group consisting of polypropylene, polyethylene, polyester, polyethylene terephthalate, polybutylene terephthalate, polyamide, polylactic acid, and combinations thereof. The first layer may comprise heat fusible fibers, which may be mixed with natural fibers in the first layer of the topsheet. The term “heat fusible fibers” means fibers that when they are heated at a certain temperature, the fibers can fusion bond to other fibers that comprise the same material or different material from the heat fusible fibers.

The first layer may comprise natural fibers, such as cotton fibers and rayon fibers, to improve the softness of the substrate, as well as to increase the amount of biodegradable material used. Using natural fibers for layers on the wearer-facing side of absorbent articles is generally desired. The first layer 1 may thus comprise at least 15% by weight, or at least 30% by weight, or at least 50% by weight, or at least 60% by weight, or at least 75% by weight, or at least 95% by weight of natural fibers, such as cotton fibers, by weight of the first layer. The first layer may also be made of 99% to 100% by weight of natural fibers, such as cotton fibers, by weight of the first layer. Natural fibers may be selected from the group consisting of wheat straw fibers, rice straw fibers, flax fibers, bamboo fibers, cotton fibers, jute fibers, hemp fibers, sisal fibers, bagasse fibers, hesperaloe fibers, miscanthus, marine or fresh water algae/seaweeds and combinations thereof.

The first layer may be hydrophilic or hydrophobic. In order to have a hydrophobic first layer, a hydrophobic treatment may be applied to the first layer. The hydrophobic treatment may be based on synthetic material, at least to some extent, derived from natural sources. The hydrophobic treatment may be based on a natural compound, such as selected from the group consisting of natural oil, butters or waxes and combination thereof. Some examples, but not limited to, are cotton seed oil, Coconut oil, Avocado oil, Jojoba oil, Castor-seed oil, Soybean oil, Almond oil, Lanolin, Olive oil, Sunflower seed oil, Eucalyptus oil, Shea butter, Cocoa butter, Murumuru butter, Almond butter, Avocado butter, Aloe butter, Mango butter, Beeswax, Soy wax, Candelilla wax, Rice-bran wax, Coconut wax.

Second Layer

The substrate disclosed herein, referring to FIGS. 1 and 2, comprises a second layer 2 in a face to face relationship with the first layer 1, as described above. The second layer 2 may be any type of web conventionally used in the art. For example, it may be a nonwoven web of natural fibers, synthetic fibers or a combination of natural and synthetic fibers. Alternatively, the second layer may be a polymeric film as is commonly used in backsheet. Typically, the polymer film may be breathable or not, but is liquid impermeable.

The list of synthetic fibers and of natural fibers corresponds to the list disclosed above for the first layer. Typically, the synthetic fibers are selected from the group consisting of polypropylene, polyethylene, polyester, polyethylene terephthalate, polybutylene terephthalate, polyamide, polylactic acid, and combinations thereof. The synthetic fibers may be single component fibers, multi-component fibers such as bicomponent fibers and combinations thereof. The fibers may have any suitable deniers or denier ranges and/or fiber lengths or fiber length ranges. The fibers in the nonwoven web can then be bonded via spunlacing processes, hydroentangling, calendar bonding, through-air bonding and resin bonding. The second layer may typically have a basis weight of from 5 g/m² to 50 g/m², or from 7 g/m² to 30 g/m², or from 7 g/m² to 20 g/m².

Several examples of nonwoven materials suitable for use as a second layer include, but are not limited to, those disclosed above with respect to the first layer.

Apertures

The substrate of the present invention optionally comprises a plurality of apertures. In some embodiments, the first layer of the substrate comprises a plurality of apertures 5. The second layer of the substrate may have a plurality of apertures 5 at least partially, or completely aligned with the apertures 5 of the first layer 1. The apertures 5 of the first layer 1 and of the second layer 2 may thus be the same apertures. The plurality of apertures 5 of the second layer 2 may have at least partially the same width and/or length as the apertures 5 of the first layer 1.

In some embodiments, as illustrated in FIG. 2, the substrate 30 may comprise a plurality of apertures 5 extending through the first layer 1 and the second layer 2.

The apertures 5 may be typically regularly aligned according to a desired pattern.

The plurality of apertures 5 may be uniformly distributed on the first layer 1. To ensure material stability, the smallest distance between the majority of the apertures regardless of their particular shape and size may be at least 0.3 mm, or at least 1.0 mm. This distance is measured center-to-center on the first surface of the first layer of the topsheet.

The apertures 5 may extend inwardly, away from the garment-facing side and toward the absorbent core when an outer cover in an absorbent article comprises the substrate disclose herein. The amount of extension of the side walls of the apertures of the first layer and of the second layer should be at least 0.1 mm beyond the first surface of the first layer, or at least 0.2 mm beyond the first surface of the first layer. The apertures may be tapered and take a conical shape such that the diameter of the aperture is larger proximate the second surface than the diameter of the opening proximate the bottom edge of the aperture. The apertures may vary in shape. For example, the shape of the apertures as seen from the first surface of the first layer may be circular, elliptic, rectangular, or polygonal.

The apertures may also vary in size, such as a size of 4 mm² or less, of 3.5 mm² or less, of 3.0 mm² or less, or of 2.5 mm² or less. The size of the apertures may not be less than 0.2 mm², advantageously not less than 0.5 mm². The size is determined on the surface of the substrate which is most outwardly placed, so typically the first side of the first layer, and e.g. for conical apertures the larger aperture opening is determined.

### Method of Making the Three-Dimensional Substrate

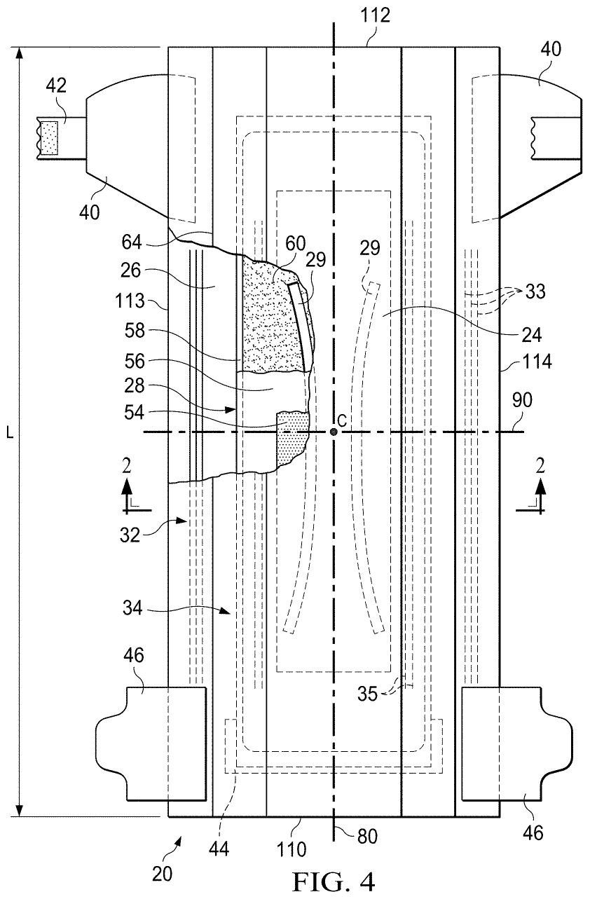

The three-dimensional substrate (with or without apertures) may be industrially produced at high speed by any known and suitable methods, for example as described in WO2017/156200 (Orr et al., P&G) and PCT/CN2018/110397, filed on Oct. 16, 2018 (Erdem et al, P&G). FIG. 3 is a schematic illustration of one example process for forming protrusions in the first layer and bonding this layer to a second layer, which is briefly discussed below. More details of this process, including photographs of the embossing rolls, can be found in PCT/CN2018/110397.

Referring to FIG. 3, a first layer material 200 may go through a pair of rolls named A and B. The first layer material 200 is to form the first layer 1 of the substrate.

The speed of the roll A and B may be from 5 to 600 meters/minute. The temperature range of the roll A may be from 40 to 200° C. The temperature range of the roll B may be from 30 to 200° C. The roll A may comprise a plurality of protrusions 201 extending radially outwardly from the roll A. The roll A may also comprise a plurality of recesses 202 formed in a radial outer surface of the roll A. The depth of the recesses 202 of the roll A may be from 0.5 to 10 mm, the height of the protrusions 201 of the roll A may be from 0.5 to 9 mm. The roll B may comprise a plurality of protrusions 203 extending radially outwardly from the roll B. The roll B may also comprise a plurality of recesses 204 formed in a radial outer surface of the roll B. The distal end of the plurality of protrusions 203 of the roll B may have the shape of a pin 205.

The protrusions 201 on the roll A may have a different size, shape, height, area, width and/or dimension than the protrusions 203 on the roll B. The recesses 202 formed in the roll A may have a different size, shape, height, area, width, and/or dimension than the recesses 204 formed in the roll B. The recesses 202 in the roll A may be configured to at least partially receive the protrusions 203 of the roll B, thereby creating the protrusions in the first layer material 200. The roll A may comprise a plurality of holes in the recesses area in order to receive the shape of pin 205 of the protrusions 203 of the roll B. Therefore, a plurality of apertures 5 are formed in the first layer material 200 between each two adjacent protrusions of the first layer material 200. The first layer material 200, after going through the roll A and the roll B may comprise a plurality of protrusions 9 and a plurality of apertures 5 between each two adjacent protrusions.

A second layer material 206 may be brought by a concave roller C. The hot-melt adhesive can be added on the first surface of the second layer material 206 by an equipment D before the second layer material 206 is in contact with the first layer material 200. The hot-melt adhesive can be advantageously uniformly sprayed on the second layer material at the basis weight indicated previously, for example 5 g/m². The roll C may comprise a plurality of holes in order to receive the shape of pin 205 of the protrusions 203 of the roll B. The second layer material 206 may pass through the roll C and the roll B and contact with the first layer material 200 at the protrusions 203 of the roll B. As the protrusions 203 of the roll B may have the shape of a pin, a plurality of apertures may be created also on the second layer material 206. The apertures in the second layer material 206 may in this way be aligned with the apertures in the first layer material 200. Pressure is applied in the land areas of the first layer and the second layer so that the adhesive forms bonds between the first and second layers to form the substrate. At the end of this process, a substrate material as illustrated in FIG. 2 comprising a three-dimensional, apertured first layer 1 in contact with a second layer 2 between the protrusions 9 of the first layer is obtained.

Penetration of at least part of fibers of the first layer into the second layer at the apertures improves the integrity of the substrate by reducing the risk of delamination of the first and second layer. Fiber penetration creates “anchor points” which stabilizes the substrate of first and second layer, even if the contact area between first and second layer is reduced as only the first layer has protrusions. The anchor points also reduce the risk of fiber fuzzing.

A substrate 30 for the invention but without apertures, as for example illustrated in FIG. 1 may be more easily made by first forming a three-dimensional first layer, typically by engaging a first layer material between a first and second forming members and mechanically deforming the material to form a first layer having a three-dimensional shape. Such methods are well known, and for example are described in WO2017/156200 and WO2017/156203, and PCT/CN2018/110397. The adhesive is then applied, e.g. by spraying, on the second layer material and the first layer material is bonded to the second layer material by applying pressure in the land areas 8, but without aperturing, to form the dual layer substrate of the invention.

In addition to using adhesive or alternatively, the first layer may be attached to the second layer in the bonding areas by embossing or by pin bonding. The term “embossing” means creating bonding points between the first layer and the second layer by heat or pressure for example.

When an absorbent article comprises the three-dimensional substrate disclosed herein, the three-dimensional substrate is disposed for form at least part of the garment-facing side of the article in such a way that the first layer is oriented outwardly relative to an absorbent article, so that the protrusions can be felt by the caretaker or a user feeling the garment-facing side of the article.

### Absorbent Article

Absorbent articles will now be generally discussed and further illustrated in the form of a baby diaper 20 as exemplarily represented in FIG. 4. FIG. 4 is a plan view of the exemplary diaper 20 in a flattened-out configuration with the taped ends opened and the garment-facing side turned up. An article that is presented to the user closed such as a training pant may also be represented flattened out by cutting it along its side waists. The diaper will typically have a front edge 110, a back edge 112 and the longitudinally-extending lateral side edges 113, 114. The front edge 110 forms the edge of the front waist and the back edge 112 of the back waist, which together when worn by the wearer form the opening for the waist of the wearer. The lateral edges 113, 114 can each form one of the leg openings. The diaper 20 notionally comprises a longitudinally centerline 80 dividing the article in a left side and a right side, and a perpendicular transversal centerline 90 disposed at half the length of the article as measured on the longitudinal centerline 80, with both centerlines crossing at the center point C. The taped back ends 42 attached on the front of the diaper to such as a landing zone 44.

Other layers of the absorbent article are better illustrated in FIG. 5, which shows in cross-section in addition to the liquid permeable topsheet 24 and the backsheet 26, an absorbent core 28 between the topsheet 24 and the backsheet 26.

A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers.

An optional acquisition and/or distribution layer (or system) 50 is represented in FIG. 5 together with other typical diaper components. The acquisition and/or distribution layer may comprise one layer or more than one layer. Typical acquisition and/or distribution layers may not comprise SAP as this may slow the acquisition and distribution of the fluid, but an additional layer may also comprise SAP if some fluid retention properties are wished.

The absorbent article may typically comprise a pair of partially upstanding barrier leg cuffs 34 having elastic elements 35 and elasticized gasketing cuffs 32 having elastic elements 33 substantially planar with the chassis. Both types of cuffs are typically joined to the chassis of the absorbent article typically via bonding to the topsheet and/or backsheet.

The absorbent article may comprise elasticized back ears 40 having a tape end 42 which can be attached to a landing zone 44 at the front of the article, and front ears 46 typically present in such taped diapers to improve containment and attachment.

The absorbent article of the present invention may meet certain parametric requirements as detailed below. Among the parameters of interest, Thickness, Qmax (Qmax-dry and Qmax-wet) and Compression Recovery Rate (“CRR”) are obtained from the FTT Test methods below. Information about the FTT Test method may be found in the paper “Fibers and Polymers 2014, Vol. 15, No. 7, 1548-1559” titled “A Simultaneous Measurement Method to Characterize Touch Properties of Textile Materials” by Xiao Liao et al.

The absorbent article of the present invention may have a relatively low Qmax-wet. The absorbent article has a Qmax-wet of about 1300 W/m² or less, or about 1250 W/m² or less, or about 1200 W/m² or less as measured according to FTT Test described herein. An outer cover comprising the three-dimensional substrate may contribute to a low Qmax-wet. Qmax indicates the maximum thermal flux transmitted during compression, without being bound by theory, the less the Qmax, the warmer the tactile perception of the material. Without being bound by theory, the void volume between the first and second layer may provide a higher thermal resistance with the substrate as the void volume contains more air which is more difficult for heat to transfer through. When the absorbent core is wet, the substrate with void volume and high thermal resilience may effectively prevent the heat transferring between the absorbent core and the most outside of the absorbent article. Due to a high thermal resilience of the substrate, touch feeling of the most outside of the absorbent article may provide a dry and pleasant feel even when the absorbent core is wet.

The absorbent article of the present invention comprises an outer cover comprising a three-dimensional substrate disclosed herein which may have a Qmax-dry of about 1050 W/m² or less, or about 1000 W/m² or less, or about 950 W/m² or less as measured according to FTT Test described herein.

The absorbent article of the present invention comprises an outer cover comprising a three-dimensional substrate disclosed herein which may have a thickness of 0.38 mm or higher, or of about 0.40 mm or higher as measured according to FTT Test described herein. Without being bound by theory, a three-dimensional substrate disclosed herein and having have a thickness of about 0.38 mm or higher may sustain load and maintain the three-dimensional structure including the void volume between the first layer and the second layer in the substrate.

The absorbent article of the present invention comprises an outer cover comprising a three-dimensional substrate disclosed herein which may have a CRR of about 0.35 or higher, or of about 0.40 or higher as measured according to FTT Test described herein. Without being bound by theory, a three-dimensional substrate disclosed herein and having a high CRR may sustain load and maintain the three-dimensional structure including the void volume between the first layer and the second layer in the substrate.

Absorbent Core

As used herein, the term “absorbent core” refers to a component used or intended to be used in an absorbent article and which comprises an absorbent material and optionally a core wrap. As used herein, the term “absorbent core” does not include the topsheet, the backsheet and any acquisition-distribution layer or multilayer system, which is not integral part of the absorbent core. The absorbent core is typically the component of an absorbent article that has the most absorbent capacity of all the components of the absorbent article. The terms “absorbent core” and “core” are herein used interchangeably.

Referring to FIGS. 4 and 5, the absorbent core 28 can absorb and contain liquid received by the absorbent article and comprise an absorbent material 60, which may be cellulose fibers, a blend of superabsorbent polymers and cellulose fibers, pure superabsorbent polymers, and/or a high internal phase emulsion foam. The absorbent core 28 may comprise absorbent material free channels 29, through which the top side 56 of the core wrap may be bonded to the bottom side 58 of the core wrap. The core wrap bonds 27 may at least persist as the absorbent core 28 swells upon liquid absorption and creates three-dimensional channels at the wearer-facing surface of the article. Of course, this is entirely optional, the absorbent core may also not have bonded channels, or even unbonded channels. The absorbent material defines an absorbent material area 8, which may be rectangular as show in in FIG. 4, but it is also common to have a shaped area which is tapered in the area around the transversal centerline 90.

The absorbent material comprises a liquid-absorbent material commonly used in disposable absorbent articles such as comminuted wood pulp, which is generally referred to as airfelt or fluff. Examples of other suitable liquid-absorbent materials include creped cellulose wadding; melt blown polymers, including co-form; chemically stiffened, modified or cross-linked cellulosic fibers; tissue, including tissue wraps and tissue laminates, absorbent foams, absorbent sponges, superabsorbent polymers (herein abbreviated as “SAP”), absorbent gelling materials, or any other known absorbent material or combinations of materials.

The absorbent material in the absorbent core can be any type. It can be an airfelt core comprising wood cellulose fibers such as pulp fibers mixed with SAP, or an airfelt-free core free from such cellulose fibers. Airfelt cores typically comprises from 40% to 80% of SAP. For absorbent cores comprising a relatively high proportion of SAP at least partially enclosed within the core wrap, the SAP content may represent in particular at least 80%, 85%, 90%, 95% and up to 100%, of superabsorbent polymer by weight of the absorbent material. The absorbent material may in particular comprise no or only small amount of cellulose fibers, such as less than 20%, in particular less than 10%, 5% or even 0% of cellulose fibers by weight of the absorbent material. The absorbent core may comprise an absorbent material comprising at least 80%, at least 90%, at least 95%, or at least 99% by weight of the absorbent core. The term “superabsorbent polymer” refers herein to absorbent material, which may be cross-linked polymer, and that can typically absorb at least 10 times their weight of an aqueous 0.9% saline solution as measured using the Centrifuge Retention Capacity (CRC) test (EDANA method WSP 241.2-05E). The SAP may in particular have a CRC value of more than 20 g/g, or more than 24 g/g, or of from 20 to 50 g/g, or from 20 to 40 g/g, or from 24 to 30 g/g. The SAP may be typically in particulate forms (superabsorbent polymer particles), but it not excluded that other forms of SAP may be used such as a superabsorbent polymer foam for example.

Backsheet

An absorbent article according to the present invention comprises a backsheet. The backsheet may be designed to prevent the exudates absorbed by and contained within the absorbent article from soiling articles that may contact the absorbent article, such as bed sheets and undergarments. The backsheet may be substantially water-impermeable. Suitable backsheet materials may include breathable materials that permit vapors to escape from the absorbent article while still preventing exudates from passing through the backsheet. The backsheet may comprise a colored graphic. The backsheet may comprise a liquid impermeable film. The backsheet may comprise a wetness indicator.

Outer Cover

An absorbent article according to the present invention comprises an outer cover forming at least part of a garment-facing surface of the absorbent article. The outer cover comprises a three-dimensional substrate disclosed herein so that a first layer of the three-dimensional substrate forms at least part of the garment-facing substrate. The three-dimensional substrate disclosed herein is disposed on the garment-facing side of the article. When a backsheet comprises a liquid impermeable polymer film, the polymer film and the substrate disclosed herein may be disposed in a face to face relationship in a such way that the substrate is towards the garment-facing side of the article, and the film is towards an absorbent core of the article. The first layer is oriented outwardly relative to the article, so that the protrusions can be felt by the caretaker or a user feeling the garment-facing side of the article.

The absorbent article may also comprise other typical components, which are not represented, such as a back-elastic waist feature, a front elastic waist feature, transverse barrier cuff(s), a lotion application, etc.

Components of the disposable absorbent article described in this specification can at least partially be comprised of bio-sourced content as described in US 2007/0219521A1 Hird et al published on Sep. 20, 2007, US 2011/0139658A1 Hird et al published on Jun. 16, 2011, US 2011/0139657A1 Hird et al published on Jun. 16, 2011, US 2011/0152812A1 Hird et al published on Jun. 23, 2011, US 2011/0139662A1 Hird et al published on Jun. 16, 2011, and US 2011/0139659A1 Hird et al published on Jun. 16, 2011. These components include, but are not limited to, topsheet nonwovens, backsheet films, backsheet nonwovens, side panel nonwovens, barrier leg cuff nonwovens, super absorbent, nonwoven acquisition layers, core wrap nonwovens, adhesives, fastener hooks, and fastener landing zone nonwovens and film bases. In at least one embodiment, a disposable absorbent article component comprises a bio-based content value from about 10% to about 100% using ASTM D6866-10, method B, in another embodiment, from about 25% to about 75%, and in yet another embodiment, from about 50% to about 60% using ASTM D6866-10, method B. In order to apply the methodology of ASTM D6866-10 to determine the bio-based content of any disposable absorbent article component, a representative sample of the disposable absorbent article component must be obtained for testing. In at least one embodiment, the disposable absorbent article component can be ground into particulates less than about 20 mesh using known grinding methods (e.g., Wiley® mill), and a representative sample of suitable mass taken from the randomly mixed particles.

### Measurement

Unless particularly mentioned, all tests are performed in a room maintained at 23±2° C. and 50±5% relative humidity, and samples are pre-conditioned in a room maintained at about 23±2° C. and about 50±5% relative humidity, for at least 2 hours prior to testing.

### 1. FTT Test

### 1-1. Sample Preparation

Three-dimensional Substrate: Lay a three-dimensional substrate flat on a bench with a technical face-side facing upward, and a 110 mm by 110 mm square shape of sample is cut using a scissor. The technical face-side is the surface intended to be used as a garment-facing surface when used in an absorbent article. Five samples are cut from an identical three-dimensional substrate, or absorbent articles from the same package and cut out from the same area of each article, for each set of measurement. Samples are pre-conditioned in a room maintained at 23±2° C. and 50±5% relative humidity, for at least 24 hours prior to testing.

Absorbent Article for Qmax-Wet

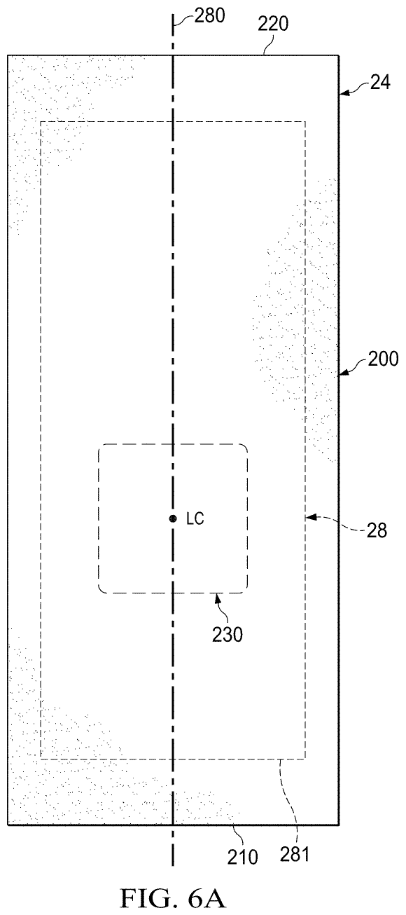

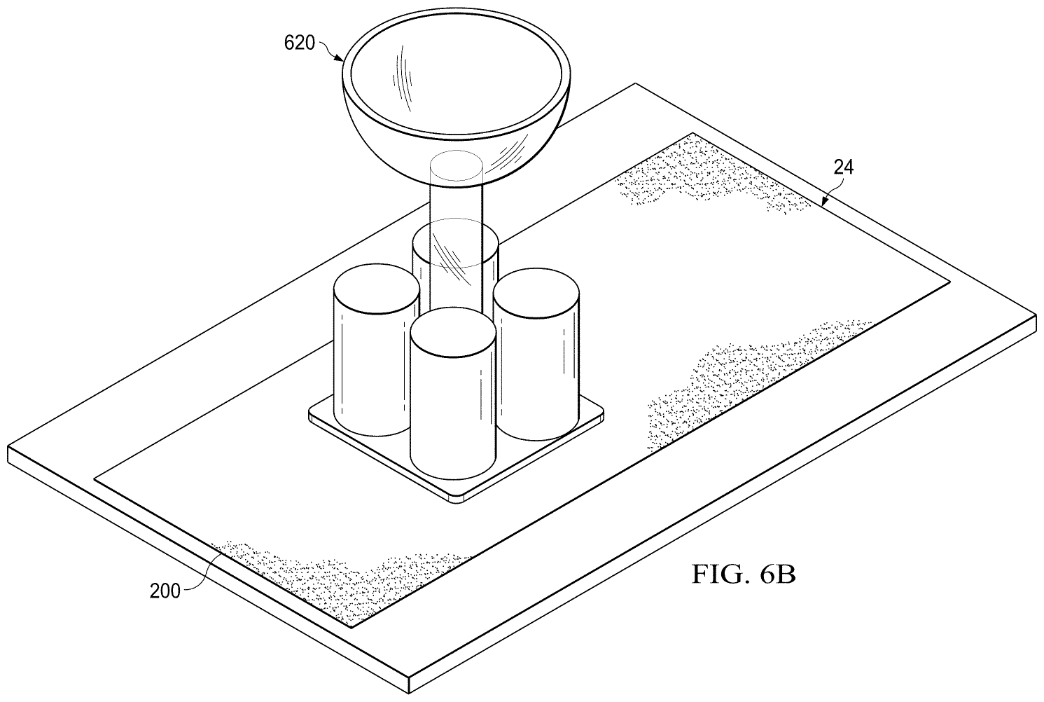

Remove front and back ears, or in the case of a pant diaper, remove belt or side panels as well. Referring to FIG. 6A, lay the cuff-removed absorbent article 200 on a bench with a topsheet 24 facing upward. Draw a longitudinal centerline 280, and determine a loading center LC of a loading location along the longitudinal centerline 280, distanced 76 mm away from a front edge 281 of an absorbent core 28. Draw a 110 mm by 110 mm square shape of testing area 230 on a backsheet side of the cuff-removed absorbent article 200 in such a way that a center of the testing area 230 corresponds to the LC.

Lay the cuff-removed absorbent article 200 on the bench with a topsheet facing upward and attach it to the bench using a hook material, adhesive tape, or the like. Place a loading tool 620 on a loading location in such a way that LC in located in the middle of the loading tube 622. The weights 624 (500 g per each) on the loading tool 620 creates ˜0.3 psi pressure. The equilibration time before dosing the first loading is 5 minutes. Load a first gush of 24 ml of 0.9% saline solution through the loading tube 622, then immediately record the time by a timer. The equilibration time between consecutive loadings is 5 minutes. Load a second gush of 24 ml of 0.9% saline solution through the loading tube 622, then immediately record the time by a timer. Wait for 5 minutes before Qmax test.

### 1-2. FTT Test

The samples are measured using the Fabric Touch Tester (FTT M293) running FTT system software (available from SDL Atlas). FTT includes five modules, which may be activated at the same time and recorded of the dynamic responses from the samples, depending on the sample. They include compression, thermal, bending, friction, and surface modules. The instrument is calibrated according to the manufacturer's instructions using the standard calibration fabric provided by the vendor. All testing is performed in a room maintained at 23±2° C. and 50±5% relative humidity.

The test procedures are conducted according to the Operating Instructions for the FTT M293 manual. The sample with technical face-side upward is placed on the lower plate. Put the 110 mm by 110 mm square shape of sample on the compression lower plate for the Compression and Thermal tests. The Compression and Thermal tests are initiated with single surface testing mode and the sample would be pushed downwards by the upper plate applying a continuously increasing normal force from 0-8470 gf (i.e. 0-70 gf/cm²).

Five samples are measured, and test parameters, or any subset thereof, are calculated and reported with the average value.

| Item | Module | Parameter | Symbol | Unit |
|---|---|---|---|---|
| 1 | Compression | Thickness | T* | mm |
| 2 | Compression | Compression/Recovery Ratio | CRR | |
| 3 | Thermal | Thermal Maximum Flux | Qmax | W/m² |

Thickness (T): This parameter denotes measured thickness under a pressure of 41 gf/cm².

1-1. Thickness Test
Sample thickness (T) is the measured thickness during compression under a pressure of 41 gf/cm².

1-2. Compression Recovery Rate Test

This parameter denotes a ratio of the recovery work (no data need?) to compression work. CRR is obtained according to equation (2) wherein Da is the initial thickness at zero pressure, Dc is minimum thickness at maximum pressure, P is the measured pressure and D is the measured thickness during compression, P′ is the measured pressure and D′ is the measured thickness during recovery.

$$CRR = \frac{{\int }_{D}^{D} \text{?} \text{?} P d D}{{\int }_{D}^{D} \text{?} \text{?} \text{?} d {D}^{′}} \\ \text{?} \text{indicates text missing or illegible when filed} \qquad ( 2 )$$

1-3. Qmax Test

Thermal Maximum Flux (Qmax) denotes the maximum thermal flux measured according to formula (1) wherein H is the measured heat flux.

Q max=max(H) (1)

### 2. Dampness Difference Test

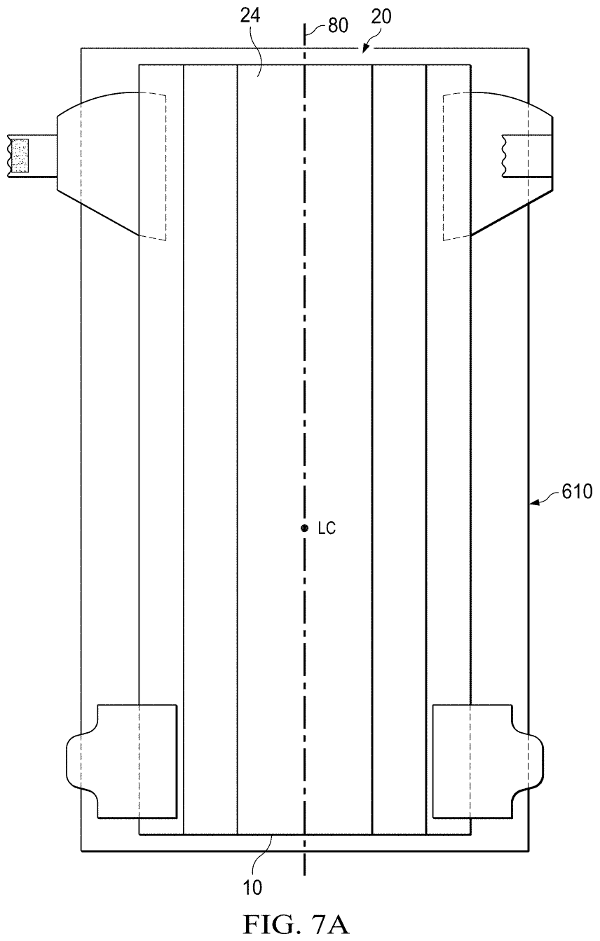

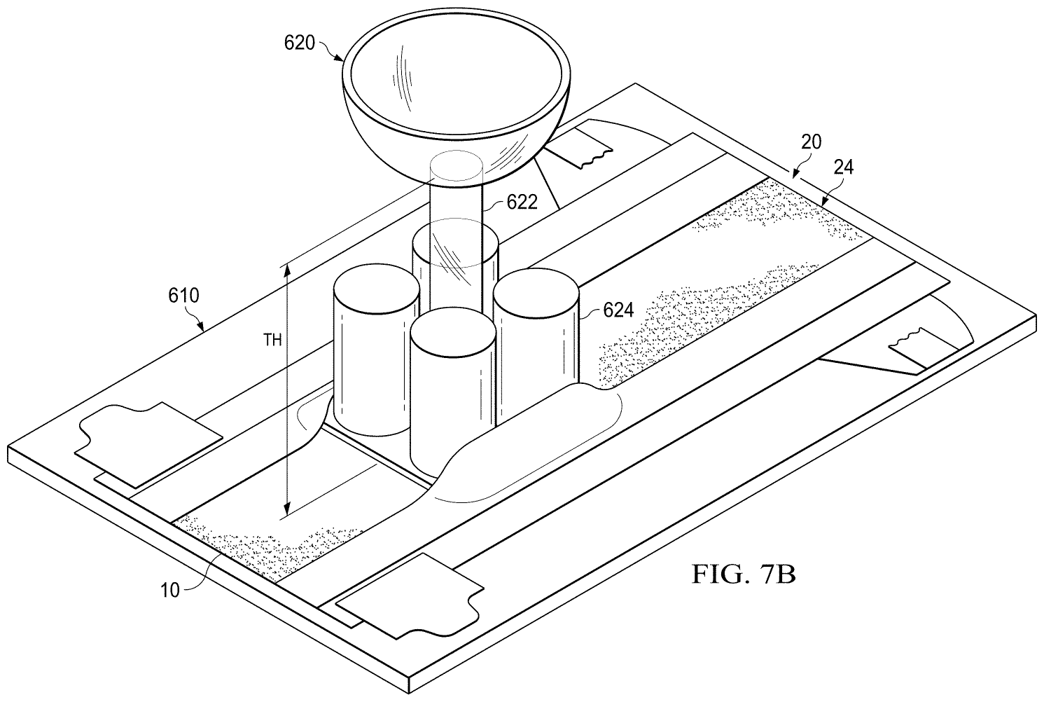

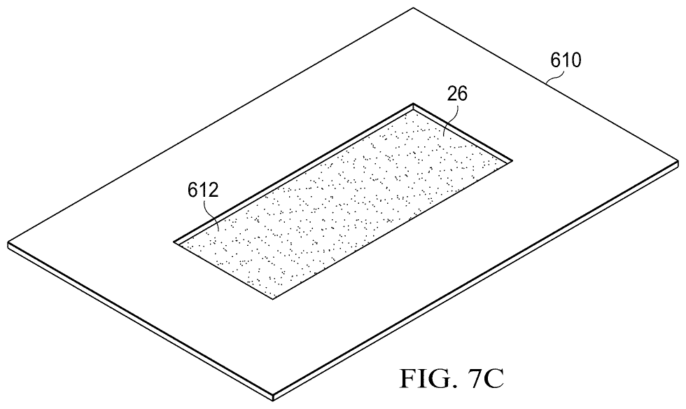

Referring to FIG. 7A-7C, lay a sample absorbent article 20 on a bench plate 610 (25 cm by 40 cm) having a 110 mm by 210 mm hollow part 612 in the middle of the plate with a topsheet 24 facing upward. Attach a front edge side and a back edge side of the absorbent article 20 to the plate 610 using a hook material, adhesive tape or the like. Draw a longitudinal centerline 80. The loading center LC of a loading location is along the longitudinal centerline 80, and distanced 12 cm away from a front edge 10 of the absorbent article 20.

Place a loading tool 620 on top of loading location in such a way that the loading center LC is located in the middle of the loading tube 622. The weights 624 (500 g per each) on the loading tool 620 creates ˜0.3 psi pressure. The equilibration time before dosing the first loading is 5 minutes. Load a first gush of 24 ml of 0.9% saline solution through the loading tube 622, then immediately record the time by a timer. The equilibration time between consecutive loadings is 5 minutes. Load a second gush of 24 ml of 0.9% saline solution through the loading tube 622, then immediately record the time by a timer. Wait for 5 minutes before sensory evaluation.

Remove the loading tool 620 from the absorbent article 20, and turn over the bench plate 610 to make the absorbent article 20 with the backsheet facing upward. Put a palm directly on the testing area in the backsheet corresponding to the loading area in the topsheet 24, with slight pressure (0.7-1 psi, 1700-2500 g in 36 cm²) for 3-4 seconds and feel the cooling feeling. Rank the test samples from more cooling feeling to less cooling feeling.

### EXAMPLES

### Nonwoven

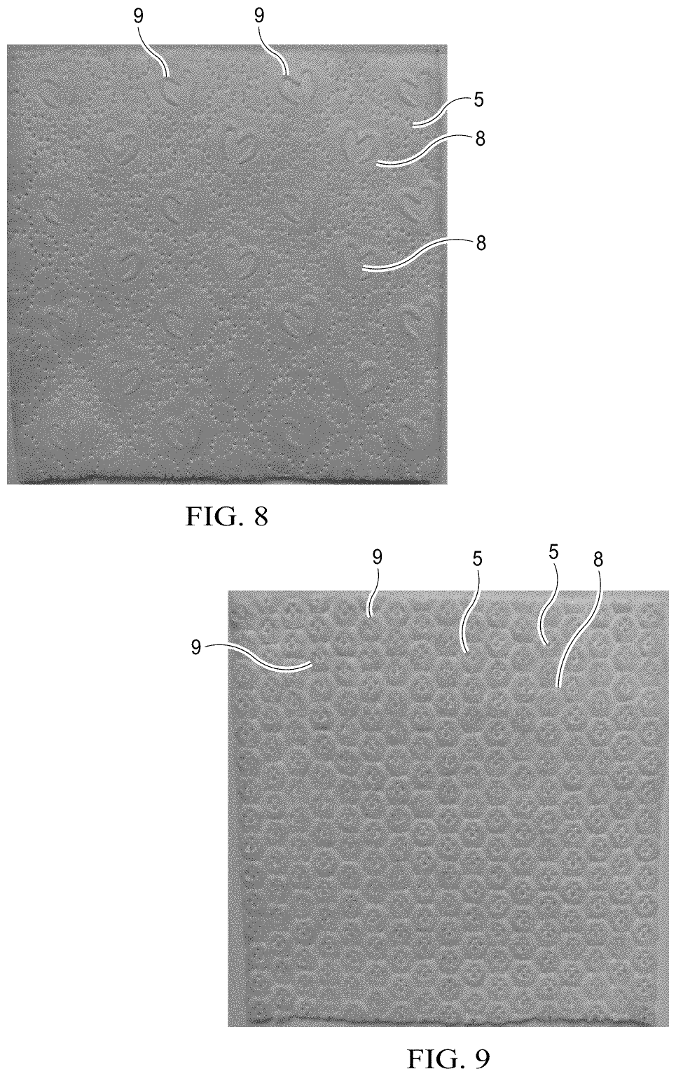

Nonwoven sample 1 as an example of three-dimensional substrates disclosed herein was prepared as below. A first layer material of 21 gsm carded air-through bonded nonwoven comprising PE/PET fibers, and a second layer material of 21 gsm carded air through nonwoven made of PE/PET fibers were processed according to a process as illustrated in FIG. 5 or similar one to obtain an apertured three-dimensional nonwoven as an example of the substrate disclosed herein as illustrated in FIG. 2 with the pattern of FIG. 8 having protrusions 9, apertures 5 and one or more lands 8.

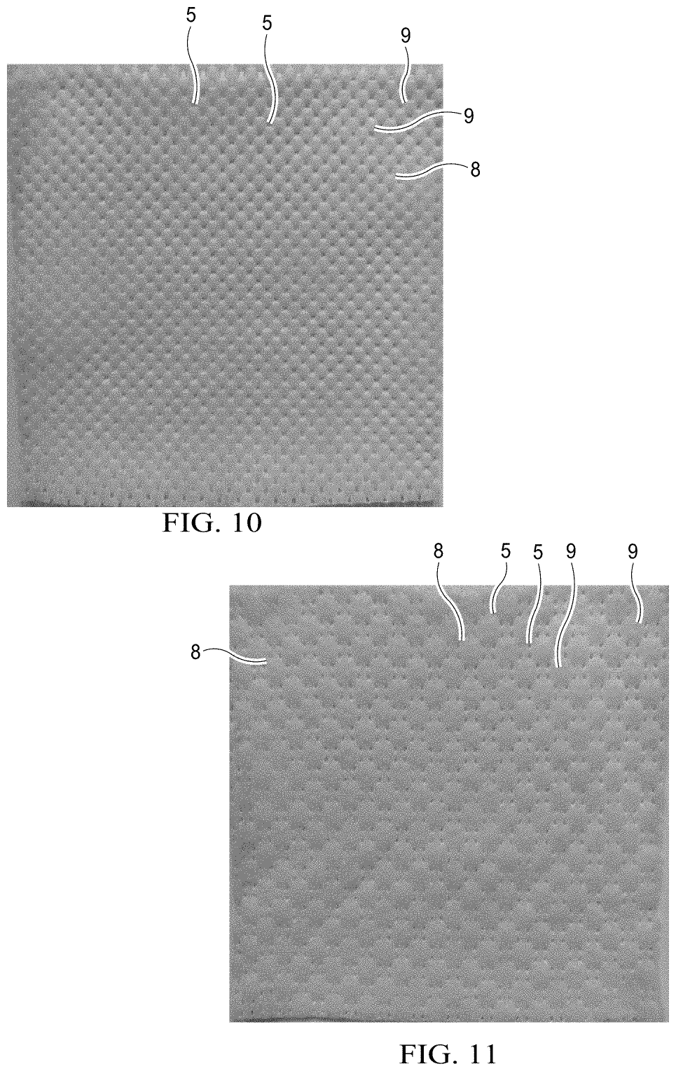

Nonwovens 2 and 3 were prepared using first layer materials and second layer materials according to table 1 below, and by processing as illustrated in FIG. 5 to obtain apertured three-dimensional nonwovens as illustrated in FIG. 2 with the patterns of FIG. 9 and FIG. 10 having protrusions 9, apertures 5 and one or more ands 8, respectively.

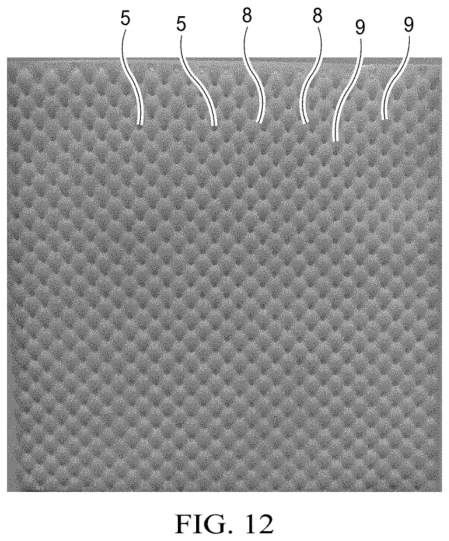

Nonwovens 4 and 5 were prepared using first layer materials and second layer materials according to table 1 below, to obtain three-dimensional nonwovens with the patterns of FIG. 11 and FIG. 12 having protrusions 9, apertures 5 and one or more lands 8, respectively. Nonwovens 4 and 5 do not have a void volume between a first layer and second layer.

Thickness, and CRR, and Qmax-dry of nonwovens were measured according to FTT Test under the MEASUREMENT section above for different nonwoven samples and illustrated in Table 1, respectively, below.

TABLE 1

| | Nonwoven 1 | Nonwoven 2 | Nonwoven 3 | Nonwoven 4 | Nonwoven 5 |
|---|---|---|---|---|---|
| first layer | 50% 1.2 dpf | 4 dpf | 4 dpf | 4 dpf | 4 dpf |
| material | PE/PET and | PE/PET, | PE/PET, | PE/PET, | PE/PET, |
| | 50% 2 dpf | 22 gsm | 22 gsm | 15 gsm | 15 gsm |
| | PE/PET, | | | | |
| | 21 gsm | | | | |
| second layer | 2 dpf | Spunbond | Spunbond, | 4dpf | 4 dpf |
| material | PE/PET, | 8 gsm, | 8 gsm | PE/PET, | PE/PET, |
| | 21 gsm | adhesive | adhesive, | 20 gsm | 20 gsm |
| | Adhesive, | 3 gsm | 3 gsm | | |
| | 3 gsm | | | | |
| Basis weight | 45 | 33 | 33 | 35 | 35 |
| (g/m²) | | | | | |
| Thickness (mm) | 0.462 | 0.408 | 0.374 | 0.292 | 0.278 |
| CRR | 0.54 | 0.40 | 0.32 | 0.50 | 0.39 |
| Qmax-dry (W/m²) | 821 | 943 | 1061 | 1161 | 1180 |

### Absorbent Articles

Diapers as exemplary absorbent articles containing an outer cover comprising a nonwoven layer were prepared using Pampers Hajimeteno Hadaeno Ichiban (Procter and Gamble Japan K.K. Japan). The outer most nonwoven layer of Pampers Hajimeteno Hadaeno Ichiban was separated from a backsheet film using techniques such as applying “Quik-Freeze®” type cold spray, or other suitable methods that do not permanently alter the properties of the sample, and replaced with nonwovens 1-5 prepared above, respectively.

Qmax of diapers was measured according to Qmax Test in the MEASUREMENT section above for different diapers in a wet state (“Qmax-wet”) and illustrated in Table 2 below.

TABLE 2

| | Diaper 1 | Diaper 2 | Diaper 3 | Diaper 4 | Diaper 5 |
|---|---|---|---|---|---|
| Outer most layer | Nonwoven 1 | Nonwoven 2 | Nonwoven 3 | Nonwoven 4 | Nonwoven 5 |
| Qmax-wet | 1080.2 (37)*, | 1261.9(60)*, | 1385 (44)*, | 1419.0(160)*, | 1460 (99) *, |
| (W/m²) | n = 5 | n = 5 | n = 2 | n = 5 | n = 5 |

*standard deviation
n: number of replicates of each diaper sample

### Dampness Difference

Dampness of diapers 1, 3 and 4 were evaluated according to Dampness Test described in the MEASUREMENT section above with 5 trained sensory panelists. Diaper 1 was ranked the least cooling feel by all 5 panelists. 4 panelists ranked diaper 3 had less cooling feeling than diaper 4, and one remaining panel could not sense the difference between diaper 3 and diaper 4.

With the same panelists, degree of differences (“DOD”) between diapers 1, 2 and 4 were also evaluated using in scale of 1-5 below, and results is indicated in Table 3.

| | |
|---|---|
| 5 | Outside normal range. Large dampness differences. I can detect a predominant |
| | note in the test option that is totally unusual material compared with control |
| | option. |
| 4 | Significant difference, can describe the moderate or large difference. I can detect |
| | a significant tactile difference to the control option. The test option no longer |
| | represents the similar tactile performance and out of expected range. |
| 3 | Noticeable difference, can describe difference with reasonable certainty. I can |
| | detect a noticeable difference in some tactile attributes. However the test option |
| | still represents the similar tactile performance vs. control option. |
| 2 | I can detect a slight difference in some tactile attributes. However, the test option |
| | still represents the similar tactile performance vs. control option. |
| 1 | No differences between the test option and the control option in terms of every |
| | tactile attributes. The test sample is equal to the control. |

TABLE 3
DOD

| | Diaper 1 vs. | Diaper 1 vs. | Diaper 2 vs. |
|---|---|---|---|
| | diaper 4 | Diaper 2 | Diaper 4 |
| DOD score | 3 | 3 | 3 |

Diaper 1 and Diaper 2 were evaluated as noticeably different in dampness feel and cooling feel over Diaper 4.

### Examples/Combinations

A. An absorbent article having a wearer-facing surface and a garment-facing surface opposite to the wearer facing surface, the absorbent article comprising
   a liquid pervious topsheet,
   a liquid impermeable backsheet,
   an absorbent core comprising an absorbent material, the absorbent core being disposed between the topsheet and the backsheet, and
   an outer cover forming at least part of the garment-facing surface, the outer cover comprising a three-dimensional substrate,
   wherein the three-dimensional substrate comprises a first layer, a second layer, and a plurality of protrusions where the first layer is unbonded to the second layer,
   wherein the outer cover is joined to the backsheet such that the first layer forms at least part of the garment-facing surface, and
   wherein the absorbent article has a Qmax-wet of about 1300 W/m² or less as measured according to Qmax Test described herein.
B. The absorbent article according to Paragraph A, wherein the three-dimensional substrate comprises a plurality of apertures in at least one of the first layer and the second layer.
C. The absorbent article according to Paragraph B, wherein the plurality of apertures extend through the first layer and the second layer.
D. The absorbent article according to any of Paragraphs A-C, wherein the three-dimensional substrate has a basis weight in the range of from about 15 gsm to about 80 gsm.
E. The absorbent article according to any of Paragraphs A-D, wherein the first layer and the second layer are bonded to each other by an adhesive.
F. The absorbent article according to any of Paragraphs A-E, wherein the first layer comprises nonwoven.
G. The absorbent article according to any of Paragraphs A-F, wherein the three-dimensional substrate comprises natural fibers.
H. The absorbent article according to any of Paragraphs A-G, wherein the three-dimensional substrate has a Compression Recovery Rate no less than about 0.35 as measured according to FTT Test.
I. The absorbent article according to any of Paragraphs A-H, wherein the three-dimensional substrate has a Thickness of about 0.38 mm or higher as measured according to FTT Test.
J. The absorbent article according to any of Paragraphs A-I, wherein the three-dimensional substrate has a Qmax-dry of about 1050 W/m² or less as measured according to FTT Test.
K. The absorbent article according to any of Paragraphs A-J, wherein the backsheet comprises film layer.
L. The absorbent article according to any of Paragraphs A-K, wherein the backsheet comprises colored graphic.
M. The absorbent article according to any of Paragraphs A-L, wherein the absorbent core comprises an absorbent material selected from the group consisting of airfelt, superabsorbent polymers and a combination thereof.
N. The absorbent article according to any of Paragraphs A-M, wherein the absorbent core comprises at least 80% of superabsorbent polymers by weight of the absorbent material.
O. The absorbent article according to any of Paragraphs A-N, wherein the absorbent core comprises a channel.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An absorbent article having a wearer-facing surface and a garment-facing surface opposite to the wearer facing surface, the absorbent article comprising:
a liquid pervious topsheet,
a liquid impermeable backsheet,
an absorbent core comprising an absorbent material, the absorbent core being disposed between the topsheet and the backsheet, and
an outer cover forming at least part of the garment-facing surface, the outer cover comprising a three-dimensional substrate,
wherein the three-dimensional substrate comprises a first layer, a second layer, and a plurality of protrusions where the first layer is unbonded to the second layer,
wherein the outer cover is joined to the backsheet such that the first layer forms at least part of the garment-facing surface, and
wherein the absorbent article has a Qmax-wet of about 1300 W/m² or less, as measured according to Qmax Test.

2. The absorbent article of claim 1, wherein the three-dimensional substrate comprises a plurality of apertures in at least one of the first layer and the second layer.

3. The absorbent article of claim 2, wherein the plurality of apertures extend through the first layer and the second layer.

4. The absorbent article of claim 1, wherein the three-dimensional substrate has a basis weight in the range of from about 15 gsm to about 80 gsm.

5. The absorbent article of claim 1, wherein the first layer and the second layer are bonded to each other by an adhesive.

6. The absorbent article of claim 1, wherein the first layer comprises a nonwoven.

7. The absorbent article of claim 1, wherein the three-dimensional substrate comprises natural fibers.

8. The absorbent article of claim 1, wherein the three-dimensional substrate has a Compression Recovery Rate of no less than about 0.35, as measured according to FTT Test.

9. The absorbent article of claim 1, wherein the three-dimensional substrate has a Thickness of about 0.38 mm or higher, as measured according to FTT Test.

10. The absorbent article of claim 1, wherein the three-dimensional substrate has a Qmax-dry of about 1050 W/m² or less, as measured according to FTT Test.

11. The absorbent article of claim 1, wherein the backsheet comprises a film layer.

12. The absorbent article of claim 1, wherein the backsheet comprises a colored graphic.

13. The absorbent article of claim 1, wherein the absorbent core comprises an absorbent material selected from the group consisting of airfelt, superabsorbent polymers and a combination thereof.

14. The absorbent article of claim 1, wherein the absorbent core comprises at least 80% of superabsorbent polymers by weight of the absorbent material.

15. The absorbent article of claim 1, wherein the absorbent core comprises a channel.

16. An absorbent article having a wearer-facing surface and a garment-facing surface opposite to the wearer facing surface, the absorbent article comprising:
a liquid pervious topsheet,
a liquid impermeable backsheet,
an absorbent core comprising an absorbent material, the absorbent core being disposed between the topsheet and the backsheet, and
an outer cover forming at least part of the garment-facing surface, the outer cover comprising a three-dimensional substrate,
wherein the three-dimensional substrate comprises a first layer, a second layer, and a plurality of protrusions where the first layer is unbonded to the second layer,
wherein the outer cover is joined to the backsheet such that the first layer forms at least part of the garment-facing surface, and
wherein the absorbent article has a Qmax-wet of about 1300 W/m² or less, as measured according to Qmax Test.
wherein the three-dimensional substrate comprises a plurality of apertures in at least one of the first layer and the second layer.

17. The absorbent article of claim 16, wherein the plurality of apertures extend through the first layer and the second layer.

18. The absorbent article of claim 16, wherein the backsheet comprises a colored graphic.

19. An absorbent article having a wearer-facing surface and a garment-facing surface opposite to the wearer facing surface, the absorbent article comprising:
a liquid pervious topsheet,
a liquid impermeable backsheet comprising a colored graphic,
an absorbent core comprising an absorbent material, the absorbent core being disposed between the topsheet and the backsheet, and
an outer cover forming at least part of the garment-facing surface, the outer cover comprising a three-dimensional substrate,
wherein the three-dimensional substrate comprises a first layer, a second layer, and a plurality of protrusions where the first layer is unbonded to the second layer,
wherein the outer cover is joined to the backsheet such that the first layer forms at least part of the garment-facing surface, and
wherein the absorbent article has a Qmax-wet of about 1300 W/m² or less, as measured according to Qmax Test.
wherein the three-dimensional substrate comprises a plurality of apertures in at least one of the first layer and the second layer.

20. The absorbent article of claim 19, wherein the plurality of apertures extend through the first layer and the second layer.
